(19) United States
(12) Patent Application Publication
(10) Pub. No.: US 2023/0204574 A1
(43) Pub. Date: Jun. 29, 2023

(54) RAPID, POINT OF CARE DETECTION OF NEUTRALIZING ANTIBODIES AGAINST A VIRUS

(71) Applicant: MASSACHUSETTS INSTITUTE OF TECHNOLOGY, Cambridge, MA (US)

(72) Inventors: Hojun LI, Cambridge, MA (US); Guinevere CONNELLY, Somerville, MA (US)

(21) Appl. No.: 17/996,449

(22) PCT Filed: Apr. 6, 2021
(86) PCT No.: PCT/US2021/025852

§ 371 Date: Oct. 18, 2022

Related U.S. Application Data
(60) Provisional application No. 63/060,724, filed on Aug. 4, 2020.
(60) Provisional application No. 63/014,886, filed on Apr. 24, 2020.

(51) Int. Cl. G01N 33/543; G01N 33/569; G01N 33/68

(52) U.S. Cl. CPC G01N 33/54388; G01N 33/56983; G01N 33/6857; G01N 2333/165; G01N 2469/20

(57) ABSTRACT

Described are point of care tests to detect circulating neutralizing antibodies against SARS-CoV-2 or another virus in a sample obtained from patients. The tests comprise lateral flow test strips and methods of use thereof.

### RELATED APPLICATIONS

This application claims the benefit of U.S. Provisional Application Ser. No. 63/014,886 filed Apr. 24, 2020 and U.S. Provisional Application Ser. No. 63/060,724 filed Aug. 4, 2020. The entire contents of the above applications are incorporated by reference herein.

### BACKGROUND OF THE INVENTION

Coronavirus disease 2019 (COVID-19) is a worldwide pandemic caused by Severe Acute Respiratory Syndrome coronavirus 2 (SARS-CoV-2) infection. Current SARS-CoV-2 testing utilizes RT-PCR of respiratory tract samples to detect virus-specific sequences.^{1,2} This approach may have suboptimal sensitivity,³⁻⁵ and has, in the past, been limited by prolonged reporting of results and widespread unavailability of reagents.⁶ Although point-of-care viral antigen testing potentially ameliorates some of these difficulties,⁷ there are ongoing shortages of materials required to obtain diagnostic samples, and direct antigen testing cannot identify previously infected individuals who have developed immunity. Recognition of individuals with either asymptomatic infection, or protective immunity from prior infection or vaccination, is critically important for allocating of healthcare worker resources, guiding social distancing practices, and limiting resource depletion from re-testing. Given the prior characteristics of the 2003 SARS epidemic, monitoring of healthcare worker serological status is crucial^{8,9} for an effective response to the current pandemic, but current resource limitations restrict testing to the acutely ill. Thus, for public health efforts to fully succeed in controlling the COVID-19 pandemic, it is critical to develop cost-effective, scalable, and widely available methods for rapidly detecting SARS-CoV-2 infection or immunity thereto.

In addition, with the advent of COVID-19 vaccines, there will be a need to monitor neutralizing antibody titer of vaccinated individuals, to provide longitudinal monitoring of antibody response in vaccinated individuals, as well as a need to quickly confirm vaccination status.

### SUMMARY OF THE INVENTION

Provided are rapid, point of care (e.g. bedside) tests to detect circulating neutralizing antibodies against SARS-CoV-2 (the virus causing the current COVID-19 pandemic) in the blood of patients. The tests comprise lateral flow test strips and methods of use thereof. The lateral flow test strips and methods use a lateral flow test strip for detecting the presence of neutralizing antibodies (nAbs) to SARS-CoV-2 in a biological sample from a patient, wherein the test strip comprises a sample application region, a test line (also referred to herein as the “non-neutralizing line”) comprising at least one angiotensin II converting enzyme type 2 (ACE2) receptor protein or fragment thereof immobilized at the test line; and optionally one or more control lines capable of binding to a control protein and/or a control protein tag in the sample. The ACE2 receptor protein fragment can, for example, be the extracellular domain of the human angiotensin II converting enzyme type 2 (ACE2) or a fragment thereof. The sample is contacted with a labeled SARS-CoV-2 antigen before or after application to the sample application region thus forming a treated sample, and wherein the strip is configured such that the presence or titer of nAbs is inversely proportional to the amount of the first detectable label captured at the test line. The biological sample can be contacted with the SARS-CoV-2 antigen on the strip itself; for example, the strip can comprise a conjugation region downstream from the sample application region but upstream of the test line, wherein the conjugation region comprises the soluble (or mobilizable) labeled SARS-CoV-2 antigen. The biological sample can alternatively or additionally be contacted with the labeled SARS-CoV-2 antigen before application to the sample application region; for example, the biological sample can be contacted with the labeled SARS-CoV-2 antigen for a time sufficient to permit binding between the SARS-CoV-2 antigen and any nAbs in the biological sample thus forming a treated sample and the treated sample is then applied to the sample application region. As used herein, a “treated sample” is a sample which has been contacted with a labeled SARS-CoV-2 antigen and optionally, a labeled antibody against a control protein.

The label of the labeled SARS-CoV-2 antigen is preferably a label that is detectable to the naked eye. Such labels include, but are not limited to, colloidal gold nanoparticles. The SARS-CoV-2 antigen can comprise all or a portion of the spike protein. For example, the SARS-CoV-2 antigen can comprise all or a portion of the receptor binding domain (RBD) of the spike protein.

A control line that binds a control protein can, for example, comprise an immobilized antibody that binds to the control protein. The control protein can, for example, be a protein that is naturally present in the biological sample, for example, at relatively stable or consistent amounts. A non-limiting example of such a plasma protein is albumin. Other non-limiting examples are Factor V and Factor IX. The biological sample can be contacted with a labeled antibody with antigenic specificity for the control protein in addition to the labeled SARS-CoV-2 antigen. This contact can occur before or after application of the sample to the sample application region. For example, the labeled antibody with antigenic specificity for the control protein can be present at the conjugation region and thus is contacted with the sample when the sample passes through the conjugation region. In another example, the labeled antibody with antigenic specificity for the control protein is part of the treated sample (that is applied to the sample application region). In certain aspects, the labeled antibody with antigenic specificity for the control protein (e.g., an antibody with antigenic specificity for albumin) is contacted with the biological sample before, after, or simultaneously with the SARS-CoV-2 antigen but before application to the sample application region. The control protein binds to the immobilized antibody at the control line and can be detected by the label of the labeled antibody (that is bound to the control protein).

The invention also includes a diagnostic kit comprising a test strip described herein and further comprising a labeled SARS-CoV-2 antigen. The kit can optionally comprise an antibody with antigenic specificity for a control protein, wherein the antibody is labeled with a second detectable label and/or a sample collection device.

The invention further encompasses a method of detecting the presence or titer of neutralizing antibodies (nAbs) to SARS-CoV-2 in a biological sample of a patient, comprising the steps of: contacting the biological sample with a SARS-CoV-2 antigen for a time sufficient for binding of nAbs present in the sample to the SARS-CoV-2 antigen thus forming a treated sample, wherein the SARS-CoV-2 antigen is labeled with a first detectable label; applying the treated sample to the sample application region of a test strip described herein so as to permit flow to the test line and optionally thereafter to the control line; and detecting the first detectable label at the test line wherein the amount of first detectable label at the test line is inversely proportional to the titer of nAbs in the sample. In additional aspects, the method comprises contacting the biological sample with a second antibody that binds to a control protein in the biological sample, wherein the antibody is labeled with a second detectable label, measuring the second detectable label at the control line, wherein the ratio of the amount of first detectable label at the test line to the amount of second detectable label at the control line is inversely proportional to the titer of nAbs in the sample. The control line can, for example, comprise an immobilized second antibody that binds to the control protein (the immobilized antibody is also referred to herein as the “first antibody”).

The invention additionally includes a method for detecting the presence or titer of neutralizing antibodies (nAbs) to SARS-CoV-2 in a biological sample of a patient, wherein the test strip comprises a conjugation region which comprises a SARS-CoV-2 antigen, wherein the SARS-CoV-2 antigen is labeled with a first detectable label and optionally wherein the conjugation region further comprises an antibody that binds to a control protein in the biological sample (also referred to herein as the “second antibody”), wherein the second antibody is labeled with a second detectable label; applying the sample to the sample application region of a test strip described herein so as to permit flow to the conjugation region and then the test line and optionally thereafter to the control line; and detecting the first detectable label at the test line wherein the amount of first detectable label at the test line is inversely proportional to the titer of nAbs in the sample. In additional aspects, the method comprises measuring a second detectable label at the control line, wherein the ratio of the amount of first detectable label at the test line to the amount of second detectable label at the control line is inversely proportional to the titer of nAbs in the sample. The control line can, for example, comprise an immobilized antibody that binds to a control protein (the immobilized antibody is also referred to herein as the “first antibody”).

In additional aspects, the lateral flow test strip comprises a conjugation region wherein the conjugation region is impregnated with a SARS-CoV-2 antigen that is labeled with a detectable label and wherein the control line is capable of detecting one or more protein tags. In certain aspects, the lateral flow method of the invention comprises a lateral flow test strip for detecting the presence of neutralizing antibodies to SARS-CoV-2 in a blood or serum sample from a patient, wherein the test strip comprises: a sample application region; a conjugation region wherein the conjugation region impregnated with at least one protein derived from SARS-CoV-2 optionally fused to a protein tag wherein the fusion protein further comprises at least one detection label that is visible to the naked eye; a test line (also referred to herein as the “non-neutralizing line”) comprising at least one extracellular domain of the human angiotensin II converting enzyme type 2 (ACE2) immobilized at the test line; and one or more control lines (also referred to herein as the “neutralizing line”) capable of specifically binding the detection label or the optional protein tag.

### BRIEF DESCRIPTION OF THE DRAWINGS

FIG. 1 is a schematic of an embodiment of the lateral flow test for detecting neutralizing antibodies against a virus in accordance with the invention. The test strip comprises a conjugation area comprising the labeled SARS-CoV-2 antigen that binds to the neutralizing antibodies and a labeled antibody against the control protein and/or the sample is pre-mixed with the labeled SARS-CoV-2 antigen and the labeled antibody against the control protein prior to application to the test strip. The test strip further includes a test line that detects non-neutralized SARS-CoV-2 antigen (antigen that is bound to neutralizing antibodies) and a control line. The control line detects a plasma control protein, for example.

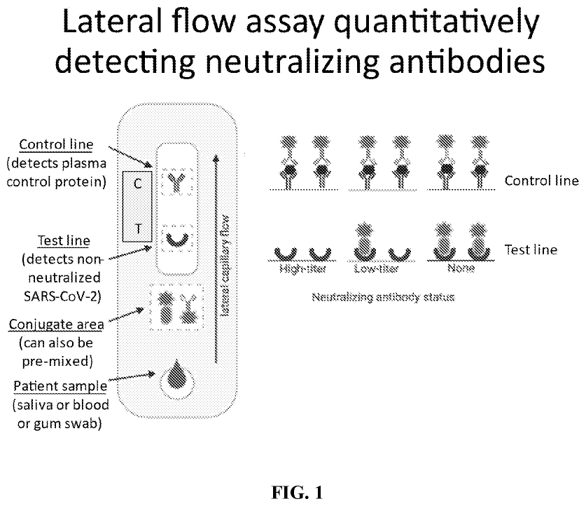

FIG. 2 is a schematic of an embodiment of the lateral flow assay for detecting neutralizing antibodies against a virus in accordance with the invention. Any neutralizing antibody in the sample can bind to the SARS-CoV-2 antigen (e.g., SARS-CoV-2 spike full-length or RBD only) which is labeled with a colored nanoparticle and an optional protein/purification tag. The patient sample which can comprise neutralizing antibody and plasma control protein is applied to the strip (thin line at bottom of figure). The plasma control protein binds to an antibody against the plasma control protein and this antibody is color-labeled. The ACE2 extracellular domain (which is the receptor for the spike protein) is immobilized at the test line by a cellulose binding domain to which it is fused (forming a fusion protein or a conjugate). Neutralized SARS-CoV-2 antigen will not bind to the immobilized ACE2 while free (non-neutralized) labeled SARS-CoV2-antigen binds at the test line. The plasma control protein is detected at the control line.

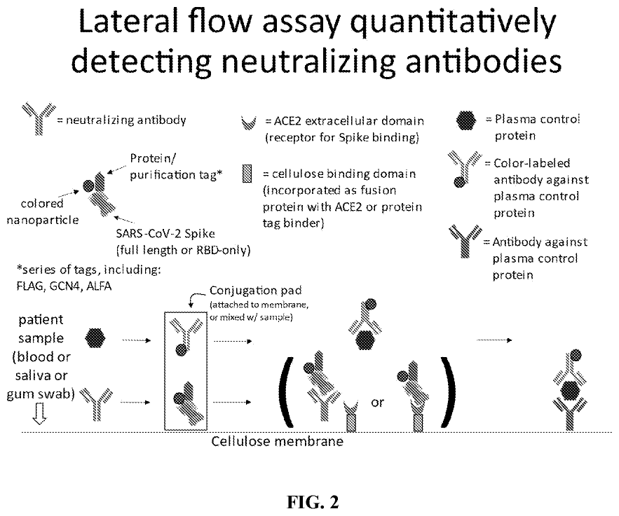

FIG. 3 is a schematic depicting exemplary quantification of neutralizing antibodies in the sample. From left to right, the first strip shows the strongest signal at the test line and a signal at the control line indicating no neutralizing antibodies in the sample. The second strip show a signal at the test line that is less strong than that of the first strip and a signal at the control line indicating the presence of some neutralizing antibody in the sample. The third strip shows a signal at the test line that is less strong than that of the second strip and a signal at the control line indicating the presence of more neutralizing antibody in the sample than that measured on the second strip. Similarly, the fourth and fifth strips show progressively stronger signals at the test line indicating the presence of more neutralizing antibody in the sample than that measured in the preceding strip. The sixth strip shows no signal at the test line and a signal at the control line indicating the presence of more neutralizing antibody than that of the fifth strip and that all or almost all of the SARS-CoV-2 antigen is neutralized (and thus unable to bind at the test line). Measurement of the ratio of the test line signal and the control line signal provides an indication of the amount neutralizing antibody present in the sample.

FIG. 4 is a diagram of an embodiment of the lateral flow test for detecting neutralizing antibodies against a virus in accordance with the invention. The control line captures soluble conjugated SARS-CoV-2 antigen that is unbound by the test line.

FIG. 5 is a diagram of an embodiment of the lateral flow test for detecting neutralizing antibodies against a virus in accordance with the invention. The one or more control lines comprise protein tag binders that bind to a protein tag that is bound to the SARS-CoV-2 antigen.

FIG. 6 is a diagram of an embodiment of the lateral flow test of the invention demonstrating the test's ability to quantify plasma neutralizing activity.

FIGS. 7A and 7B are diagrams of an example of a recombinant gene for a fusion protein including the SARS-CoV-2 spike protein or the receptor binding domain (RBD) of the spike fused to protein tags FLAG, ALFA and GCN4 and also shows purification tags in accordance with the invention. An example of an amino acid sequence corresponding to the construct comprising the full-length spike protein of SARS-CoV-2 is SEQ ID NO: 1. An example of the amino acid sequence corresponding to the construct comprising the RBD of the spike protein of SARS-CoV-2 is SEQ ID NO: 2.

FIG. 8 is a diagram of a recombinant gene for a fusion protein comprising the ACE2 extracellular domain, purification tags, and cellulose binding domain (CBD) in accordance with the invention. An example of an amino acid sequence corresponding to this construct is SEQ ID NO: 3.

FIG. 9 is a diagram of a recombinant gene for a fusion protein comprising the protein tag binder, GCN4 single chain variable fragment (scFv), purification tags, and cellulose binding domain (CBD) in accordance with the invention. An example of an amino acid sequence corresponding to this construct is SEQ ID NO: 4.

FIG. 10 is a diagram of a recombinant gene for a fusion protein comprising the protein tag binder, anti-ALFA nanobody, purification tags, and cellulose binding domain (CBD) in accordance with the invention. An example of an amino acid sequence corresponding to this construct is SEQ ID NO: 5.

FIG. 11 is a diagram of a recombinant gene for a fusion protein comprising the protein tag binder, Protein G, purification tags, and cellulose binding domain (CBD) in accordance with the invention. An example of an amino acid sequence corresponding to this construct is SEQ ID NO: 6.

### DETAILED DESCRIPTION

As used herein, the words “a” and “an” are meant to include one or more unless otherwise specified. For example, the term “a control line” means one or more control lines unless otherwise indicated.

Two major obstacles for controlling the SARS-CoV-2 infectious spread during the COVID-19 pandemic are: (1) shortage of PCR testing kits and materials for obtaining nasal swab samples, and (2) complete lack of ability to identify individuals with protective immunity who are safe to return to critical roles in society and healthcare. In addition, as more people are vaccinated against COVID-19, there will be a need to measure neutralizing antibody titer and/or detect the presence of neutralizing antibodies. Here we describe addressing the need for identifying individuals with protective immunity by producing scalable, bedside point-of-care blood tests to diagnose active SARS-CoV-2 infection and detect high-titer neutralizing antibodies.

A principal driver of uncontrolled SARS-CoV-2 infectious spread during the COVID-19 pandemic is a lack of adequate diagnostic testing availability, particularly with respect to discriminating between people with active infection, and people with immunity from prior infection. Another issue with current SARS-CoV-2 testing is speed and testing capacity; when large numbers of individuals are being tested, there can be a lag time between when the test is taken and when the test result is available. Current SARS-CoV-2 testing protocols have largely been focused on determining infectivity, based on RT-PCR of respiratory tract samples for viral RNA. However, identification of individuals with antibody-mediated protective immunity is a critical unmet need. The ability to identify such individuals is vitally important; due to their protective immunity, these individuals can safely serve in the critical healthcare and societal roles that are associated with substantial infectious risks, and can safely return to the workplace or school. Such individuals also represent a source of therapeutic convalescent serum.

In addition, as the population becomes vaccinated against SARS-CoV-2, there will be a need to monitor neutralizing antibody titer of vaccinated individuals and/or to provide longitudinal monitoring of antibody response after vaccination. There will also be a need to quickly confirm vaccination status. The test strip and method described herein can be used to accomplish these goals and specifically, to provide a measurement of antibody titer and/or detect the presence of neutralizing antibodies in a biological sample. Such biological samples included, but are not limited to, blood, serum, and abrasive gum swab. The methods described herein can be used to confirm vaccination status (whether or not an individual has been vaccinated against COVID-19) and/or to monitor titer of neutralizing antibodies after vaccination at one or more time points, e.g., providing longitudinal monitoring of antibody titer.

To rapidly detect neutralizing antibodies against SARS-CoV-2 infection at the point of care (POA or POC), we have developed a high-sensitivity lateral flow assay that that detects neutralizing antibody, for example, using one drop of patient blood, and has a read-out similar to home pregnancy tests. The assays can also measure neutralizing antibody in other sample types including, but not limited to, saliva and gum swab.

A biological sample obtained from the subject to be tested is contacted with a SARS-CoV-2 antigen that is labeled with a first detectable label. The SARS-CoV-2 antigen can be further bound to a protein tag or a purification tag. The contact between the biological sample and the SARS-CoV-2 antigen can take place on the test strip; for example, the labeled SARS-CoV-2 antigen can be present at a conjugation region or conjugation paid of the test strip which region is upstream from the test line. This contact can alternatively or additionally take place before the sample is applied to the test strip. For example, the biological sample can be contacted with the labeled SARS-CoV-2 antigen thus forming a treated sample, and the treated sample is then applied to the test strip (e.g., at the sample application region). When the biological sample is contacted with the labeled SARS-CoV-2 antigen before application to the test strip, the test strip need not comprise a conjugation region (depending, for example, on how the labeled antibody against the control protein is contacted with the sample, as discussed in more detail below).

The sample can be obtained from an animal subject. The animal subject can, for example, be a mammalian subject. Preferably the animal subject is a human subject. The subject can be an individual who had been diagnosed or was previously diagnosed with COVID-19. The subject can also be an individual that has never been diagnosed with COVID-19. The subject can be an individual that has been vaccinated against SARS-CoV-2 infection or COVID-19. The subject can be an individual suffering from “long COVID” (for example, individuals that were infected with SARS-CoV-2 but continue to experience symptoms after recovering from the initial stage of illness). The subject can also be an individual whose vaccination status is unknown. “COVID-19” and “SARS-CoV-2 infection” can be used interchangeably herein.

The SARS-CoV-2 antigen can be any portion of a SARS-CoV-2 viral protein that binds to neutralizing antibodies and to an immobilized binding partner or receptor of the SARS-CoV-2 antigen at the test line. For example, the SARS-CoV-2 antigen can be any portion or fragment a SARS-CoV-2 viral protein that binds the extracellular domain of the ACE2 receptor. In certain aspects, the SARS-CoV-2 antigen is a spike protein or a fragment thereof, such as a fragment comprising the receptor binding domain (RBD). ACE2 is the human cell surface protein that serves as the receptor to which SARS-CoV-2 RBD binds to initiate infection.

Any neutralizing antibodies present in the biological sample will bind to the SARS-CoV-2 antigen (for example, soluble RBD fragments or soluble fragments that comprise RBD), since in the body, true neutralizing antibodies will bind to the SARS-CoV-2 antigen and disrupt the virus's ability to attach to and infect human cells. The diffusion of the sample across the membrane will then carry both unbound and any antibody-bound labeled SARS-CoV-2 antigen across the membrane by lateral flow, and then reach the test line. The test line can, for example, contain or comprise an immobilized receptor (e.g., a human receptor) or a fragment thereof, or binding partner of SARS-CoV-2 antigen. A receptor for SARS-CoV2 includes for example, the ACE2 receptor or a fragment thereof. The immobilized ACE2 receptor fragment can, for example, comprise an extracellular domain of a human cell receptor of SARS-CoV-2, preferably the extracellular domain of a human ACE2 receptor. The ACE2 receptor or fragment thereof, such as the extracellular domain of the ACE2 protein, can be part of a fusion protein or conjugate comprising a moiety that binds to the test strip. For example, the fusion protein or conjugate can comprise the ACE2 receptor or fragment thereof and a cellulose binding domain that binds to the strip comprising cellulose. Engineered or modified ACE2 receptors or fragments thereof can also be used at the test line so long as they are capable of binding to the SARS-CoV-2 antigen. Thus, for example, the amino acid sequence of the ACE2 receptor can be modified and/or post-translationally modified and/or ACE2 receptor can be conjugated with another protein, peptide, or fragment. The ACE2 receptor can, for example, be modified such that it has higher affinity for RBD than wild-type ACE2. Non-limiting examples of such engineered ACE2 receptor include, for example, those described in Glasgow et al. (2020), Engineered ACE2 receptor traps potently neutralize SARS-CoV-2, PNAS 117 (45) 28046-28055 and Higuchi et al. (2020), High affinity modified ACE2 receptors protect from SARS-CoV-2 infection in hamsters, biorxiv.org/content/10.1101/2020.09.16.299891v2.article-info; the contents of each which are expressly incorporated by reference herein. ACE2 receptors and fragments thereof, modified and engineered versions thereof, including, extracellular domains of ACE2 receptors can be collectively referred to herein as “ACE2 receptor”.

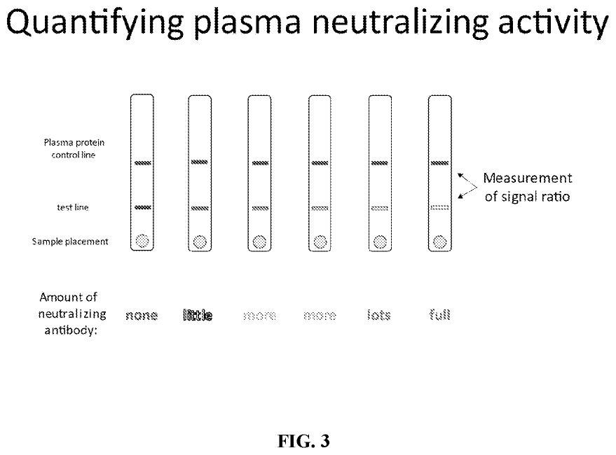

Labeled SARS-CoV-2 antigen that is not bound by neutralizing antibodies present in the sample will bind to the immobilized ACE2 receptor on the test line, causing a build-up of the label at the test line. If neutralizing antibodies are bound to the SARS-CoV-2 antigen, the labeled SARS-CoV-2 antigen will not be able to bind to the immobilized ACE2 receptor and will continue diffusing past the test line. This decreases the signal at the test line and the decrease in signal can be quantified visually (e.g., with the naked eye) or with an optical reader or a handheld computing device (such as a smartphone camera). The signal intensity at the test line is inversely correlated with neutralizing antibody titer. As used herein, the presence of nAbs is “inversely proportional” to the signal intensity when, for example, there is an absence of signal at the test line or there is a decreased signal at the test line (decreased, for example, as compared to that of a control sample comprising no nAbs). See, for example, FIG. 3. The assay and methods described herein can additionally comprise applying a control sample to the test strip, wherein the control sample is a biological sample comprising no nAbs, and measuring the signal intensity of the control sample at the test line and optionally, the control line. The optical reader or computing device can compute the intensity of the test line. Alternatively or additionally, the signal can be detected by the naked eye; for example, when a high titer of nAbs are present in the sample, the signal at the test line will be absent or will be of low intensity.

The test strip can optionally comprise one or more control test lines, wherein a binding agent for a control protein and/or a protein tag is immobilized. The term “control protein” and “protein control” are used interchangeably herein. The control protein can be a protein naturally present in the biological sample (e.g., a plasma protein). Non-limiting examples of such plasma proteins are albumin, Factor V, and Factor IX. The control protein can be detected using antibody sandwich methods that are familiar to those of skill in the art. For example, when the control protein is a plasma protein, the control line can comprise an immobilized antibody with antigenic specific for the plasma protein (also referred to herein as “an anti-plasma protein antibody” and the like). As used herein, the term “antibody” includes polyclonal and monoclonal antibodies, full-length antibodies or full length immunoglobulins, as well as antigen-binding fragments thereof, including, but not limited to, Fab, Fv and F(ab′)2, Fab′, and the like. The anti-plasma protein antibody immobilized at the control line is referred to herein as the “first” anti-plasma protein antibody or the “first” anti-albumin antibody (when the control protein is albumin). The first anti-plasma protein antibody can be a monoclonal antibody or a polyclonal antibody. In certain aspects, the first anti-plasma protein antibody or anti-albumin antibody is a polyclonal antibody. The biological sample can be contacted with a second antibody (or any other agent) with antigenic specificity for the plasma protein (either before or after application to the sample application region). The second antibody with antigenic specificity for the control protein or plasma protein (or albumin) can be contacted with the biological sample prior to application to the test strip, for example it can be part of the treated sample. Alternatively or additionally, the second antibody can be present at the conjugation region and can bind to the plasma protein or albumin in the sample as it passes through the conjugation region. The second anti-plasma protein antibody can be monoclonal or polyclonal. In certain aspects, the second anti-plasma protein antibody or second anti-albumin antibody is a monoclonal antibody. The second antibody with antigenic specificity for the plasma protein can be labeled and as such, this label can then be detected at the control line. This label can be referred to herein as the “second detectable label.” The second detectable label can for, example, be a colored nanoparticle including but not limited to colloidal gold nanoparticles. As will be understood, the strip can comprise more than one control lines that detect more than one control protein. For example, the strip can comprise a control line for detecting albumin and a control line for detecting another control protein and/or protein tag in the sample. When the control protein is a plasma protein, for example, albumin, Factor V, or Factor IX, the control protein is present in the present at a relatively constant concentration. Thus, the signal at this control line will not vary depending on the presence or absence of neutralizing antibody but will vary depending on volume of sample applied to the strip, thus serving as a control for test sample amount. When such a test is run, the ratio of signal at the test line to signal at the control line will be inversely proportional to neutralizing antibody titer. The protein/purification tag can, for example, be FLAG, GCN4 and/or ALFA, as described in more detail below.

Non-limiting examples of labels that can be used (e.g., for the first detectable label, the second detectable label or any other label used in the assay) include, but are not limited to, a colloid gold nanoparticle, a colored latex bead, a colored microparticle or nanoparticle, a magnetic particle, a carbon nanoparticle, a selenium nanoparticle, a silver nanoparticle, a fluorescent particle or nanoparticle, a quantum dot. The label can be selected from metallic particles such as gold or silver particles, or polymeric particles such as latex beads, and polystyrene particles, wherein the particles encapsulate visible or fluorescent dyes. In certain aspect, the label is visible to the naked eye. In certain aspects, the first detectable label is a gold nanoparticle. In additional aspects, the second detectable label is a gold nanoparticle. In yet additional aspects, the first detectable label and the second detectable label are each gold nanoparticles. The first and second detectable label (and other detectable labels) can be the same or different.

The SARS-CoV-2 antigen can, for example, comprise the membrane protein or a fragment thereof, the spike protein or a fragment thereof, the envelope protein or a fragment thereof, or the nucleoprotein or a fragment thereof. In certain aspects, the SARS-CoV-2 antigen comprises all or a portion of the spike protein. In yet additional aspects, the SARS-CoV-2 antigen comprises all or a portion of the receptor binding domain (RBD) of the spike protein. The SARS-CoV-2 antigen can, for example, be recombinant or recombinantly produced. The SARS-CoV-2 antigen can be a portion of an infectious agent that infects mammals, and preferably infects humans.

The test line can comprise the ACE2 protein or a fragment thereof such as the extracellular domain of an ACE2 receptor or a fragment thereof. The ACE2 protein or fragment thereof can be recombinant or recombinantly produced. In certain aspects, the immobilized ACE2 protein comprises the extracellular domain of an ACE2 receptor and optionally is recombinant or recombinantly produced. The extracellular domain of the ACE2 receptor can comprise all or a fragment of the extracellular domain of the ACE2 receptor so long that it is capable of binding to the SARS-CoV-2 antigen. The extracellular domain of an ACE2 receptor can be the extracellular domain of human ACE2 receptor; for example, a recombinantly produced human ACE2 receptor. In other embodiments, the extracellular domain of an ACE2 receptor can be the extracellular domain of a mammalian ACE2 receptor; for example, a recombinantly produced mammalian ACE2 receptor. The ACE2 receptor or fragment thereof can, for example, be immobilized at the test line by covalent coupling and/or affinity binding. For example, the ACE2 receptor can be biotinylated and can bind to the test line by biotin:streptavidin binding. In another example, the ACE2 receptor can be coupled to a cellulose binding domain, e.g., forming a fusion protein (and the strip at the test line comprises cellulose).

The biological sample can be any sample which contains neutralizing antibodies. For example, the biological sample can be a blood sample, a serum sample, or an abrasive gum swab sample.

The sample application region (a region of the test strip to which the sample or treated sample is applied) is upstream of the test line or region. The sample application region can additionally provide pH control/modification and/or specific gravity control/modification of the sample applied, and/or removal or alteration of components of the sample which may interfere or cause non-specific binding in the assay, and/or direct and control sample flow to the test region. The terms “test line” and “test region” are used interchangeably herein. When present, the conjugation region or conjugation pad is upstream of the test line and downstream of the sample application region. The conjugation region can comprise the labeled RBD (or a labeled fragment comprising the RBD or labeled SARS-CoV-2 antigen) and/or the labeled anti-control antibody in soluble or mobilizable form. The test strip can be configured such that the one or more control lines are downstream or upstream of the test line. In certain aspects, the one or more control lines are downstream of the test line. Illustrative materials material for the conjugation region (e.g., conjugation pad) include, but are not limited to, cellulose, nitrocellulose, fiberglass, cotton, woven or nonwoven paper etc. The terms “conjugation region” and “conjugate region” and “conjugation pad” are used interchangeably herein. The test strip can further comprise an absorbent region or pad at the distal end of the test strip that collects the processed sample and/or can cause the sample to move from the sample application region toward the absorbent region or pad. The test strip can also comprise a solid support which provides support for the pads and membranes of the lateral flow test strip. A buffer can be used to dilute and/or pre-treat the sample. The buffer can comprise, a salt, a mild detergent/surfactant, and an agent that inhibits or prevents non-specific binding. Exemplary buffers can, for example, comprise phosphate buffered saline (PBS) and/or saline or NaCl and non-ionic surfactants. The assay can additionally comprise a blood separation pad and/or a method to separate red blood cells and other cells or solid components from the sample. For example, a blood separation pad can be used.

The membrane used in the lateral flow immunoassay of the present invention can be made of a variety of materials which the sample to be tested can pass or move through and that are known for a person skilled in the art. For example, the materials used to form the membrane can include, but are not limited to, natural, synthetic, or naturally occurring materials that are synthetically modified, such as polysaccharides (for example, cellulose materials such as paper and cellulose derivatives as cellulose acetate and nitrocellulose); polyether sulfone; nylon; silica; inorganic materials, such as deactivated alumina, diatomaceous earth, MgSO₄, or other inorganic finely divided material uniformly dispersed in a porous polymer matrix such as vinyl chloride, vinyl chloride-propylene copolymer, and vinyl chloride-vinyl acetate copolymer; cloth, both naturally occurring (for example, cotton) and synthetic (for example, rayon); porous gels, such as silica gel, agarose, dextran, and gelatin; polymeric films, such as polyacrylamide; and the like. In a preferred embodiment, the membrane comprises nitrocellulose or cellulose. In certain embodiments, the strip comprises is paper. In some embodiments, the strip comprises cellulose, such as filter paper, chromatographic paper, nitrocellulose, and cellulose acetate.

In certain embodiments, the method comprises contacting the biological sample with a SARS-CoV-2 antigen for a time sufficient for binding of nAbs present in the sample to bind the SARS-CoV-2 antigen thus forming a treated sample, wherein the SARS-CoV-2 antigen is labeled with a first detectable label and wherein the contacting step occurs before application to the sample application region. The treated sample can further comprise a buffer, for example, a running buffer. The treated sample can be applied to the sample application region so as to permit flow of the test sample from the sample application region to the test line and optionally thereafter to the control line. When the method entails contacting the biological sample with a SARS-CoV-2 antigen prior to application to the sample application region, the test strip may or may not comprise a conjugation region. Once the sample has migrated past the test line and the optional control line, the first detectable label is detected at the test line and optionally the second detectable label is detected at the control line. The amount of first detectable label at the test line is inversely proportional to the titer of nAbs in the sample. In certain aspects, the method comprises measuring the amount of label at the test line and measuring the amount of label at the control line, wherein the ratio of the amount of first detectable label at the test line to the amount of second detectable label at the control line is inversely proportional to the titer of nAbs in the sample. The amount of detectable label at the test line or the control line can be detected with a handheld computing device including, but not limited to, a smartphone camera. In other aspects, the presence or absence of the first detectable label and optionally the presence of the second detectable label is determined visually, e.g., with the naked eye. As discussed above, the absence or low amount of first detectable label at the test line is indicative of the presence of nAbs in the sample.

In specific embodiments, the invention encompasses a rapid point-of-care lateral flow assay that quantifies neutralizing antibody titers against SARS-CoV-2 using a biological sample, such as human blood or a gingival mucosa oral swab. This test is based on brief incubation of the human test sample with recombinant SARS-CoV-2 spike protein receptor binding domain (RBD) that has been conjugated to colloidal gold. This brief incubation allows any neutralizing antibody in the sample to bind to RBD and neutralize its ability to bind to the human cell surface receptor, the ACE2 protein. The mixture is then run on a lateral flow assay membrane described herein where recombinant extracellular domain of ACE2 has been immobilized to a test line. If no neutralizing antibody is present, the gold-conjugated RBD will be captured at the test line via binding to the immobilized ACE2, thus creating a colloidal gold signal at the test line. If neutralizing antibody is present in the sample, the gold-conjugated RBD will be blocked from binding to the immobilized ACE2 extracellular domain, decreasing the signal at the test line. This decrease in signal can be quantified visually, or with a simple optical reader such as a smartphone camera. Thus, the signal intensity of the test line is inversely correlated with neutralizing antibody titer. Due to practical limitations, it may be difficult to obtain a precise volume of biological sample (e.g., blood or gingival swab) in the clinical setting. Thus, variations in the volume or amount of sample will alter the total amount of neutralizing antibody that is loaded into the test. Given this limitation, it may be advantageous to utilize an internal control for the rapid test that relies on an abundant plasma protein. One such plasma protein is human albumin, which is present in human blood and gingival swab samples, and can be used as an internal control. Other plasma proteins that can be used as an internal control include, for example, Factor V and Factor IX. In the test, in addition to the presence of gold-conjugated RBD in the brief incubation step a gold-conjugated monoclonal antibody against human albumin can also be included. Thus during the incubation the monoclonal antibody will bind to human albumin. In this lateral flow test strip, in addition to the test line that has immobilized ACE2 extracellular domain, a further away (e.g., distal or downstream) control line that has an immobilized polyclonal antibody against human albumin is included. The signal at this control line will remain constant regardless of the presence or absence of neutralizing antibody, but will vary depending on amount of sample collected, thus serving as a control for test sample amount. When the test is run, the ratio of signal at the test line to signal at the control line will be inversely proportional to neutralizing antibody titer.

In an additional specific embodiment, the gold-conjugated RBD or other SARS-CoV-2 antigen and optionally the anti-albumin monoclonal antibody are located at the conjugation region and are contacted with the biological sample when the sample flows from the sample application through the conjugate region. After the conjugation region, the sample flows to the test line where the recombinant extracellular domain of ACE2 receptor (or other ACE2 receptor) has been immobilized. If no neutralizing antibody is present, the gold-conjugated RBD will be captured at the test line via binding to the immobilized ACE2, thus creating a colloidal gold signal at the test line. If neutralizing antibody is present in the sample, the gold-conjugated RBD will be blocked from binding to the immobilized ACE2 extracellular domain, decreasing the signal at the test line. This decrease in signal can be quantified visually, or with an optical reader such as a smartphone camera. Again, the signal intensity of the test line is inversely correlated with neutralizing antibody titer. As discussed herein, the control line can comprise an immobilized polyclonal antibody against human albumin. When such a test is run, the ratio of signal at the test line to signal at the control line will be inversely proportional to neutralizing antibody titer.

The tests described herein can provide a signal or test results rapidly, for example, in less than about 60 minutes, preferably less than about 30 minutes, preferably in less than about 20 minutes and preferably in less than about 10 minutes, or less than 5 minutes. The test provides test results with an accuracy of about 90% or greater, preferably about 95% or greater, preferably about 99% or greater and preferably about 100%. The test strip can be utilized as the point-of-care. A point-of-care test is a test performed at or near the place where the sample is collected and can provide rapid results, e.g. within minutes. Such point-of-care tests can be used at the patient's bedside, at a physician's office, in an urgent care setting, at a pharmacy, at a school or university health clinic, at a long term care facility, at airports and other points of entry, and/or at other locations.

In yet other aspects, the conjugation region comprises the labeled SARS-CoV-2 antigen and the lateral flow method of the invention comprises a lateral flow test strip for detecting the presence of neutralizing antibodies to SARS-CoV-2 in a blood or serum sample from a patient, wherein the test strip comprises: a sample application region; a conjugation region wherein the conjugation region impregnated with at least one protein derived from SARS-CoV-2 optionally fused to a protein tag wherein the fusion protein further comprises at least one detection label that is visible to the naked eye; a test line (also referred to herein as the “non-neutralizing line”) comprising at least one extracellular domain of the human angiotensin II converting enzyme type 2 (ACE2) immobilized at the test line; and one or more control lines (also referred to herein as the “neutralizing line”) capable of specifically binding the detection label or the optional protein tag. In certain embodiments, we coupled our novel strategy with the preferred horse-radish peroxidase-mediated tetramethylbenzidine (TMB) visualization, which can have significantly higher sensitivity than standard colloidal gold conjugates used in other rapid assays. Thus, our test can detect high titers of neutralizing antibody in less than about 60 minutes, preferably less than about 30 minutes, preferably in less than about 20 minutes and preferably in less than about 10 minutes.

In such a test, a single drop of blood or other biological sample, (e.g., saliva or gum swab) is deposited onto one side of a test strip, preferably a cellulose or nitrocellulose membrane. The serum from the blood then diffuses across the membrane. Serum first interacts with a portion of the membrane impregnated with a viral ligand of SARS-CoV-2, preferably solubilized receptor binding domain (RBD) fragments from the SARS-CoV-2 spike protein. As described above, the RBD is responsible for the virus being able to attach to and infect human cells. The RBD fragments are preferably labeled with one or more detectable labels. Preferably the one or more detectable labels are gold nanoparticles and the horseradish peroxidase enzyme. It is understood that the full-length SARS-CoV-2 spike protein comprising receptor binding domains that target human cells for infection may also be used instead of, or in addition to, the solubilized RBD fragments of SARS-CoV-2. The full-length SARS-CoV-2 may also be labeled with a detectable label in the same manner the RBD fragments of SARS-CoV-2 are labeled.

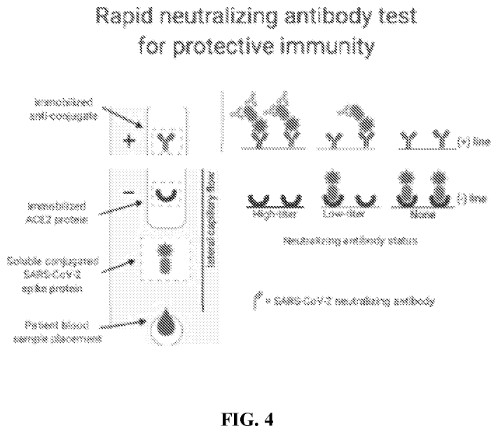

As shown in FIG. 4, any neutralizing antibodies in the patient's serum will bind to the soluble the RBD fragments, as true neutralizing antibodies will bind to the SARS-CoV-2 RBD and disrupt the virus's ability to attach to and infect human cells. The diffusion of serum across the membrane will then carry both unbound and antibody-bound labeled RBD fragments across the membrane by lateral flow, and then reach the test line.

In one embodiment, the test line contains immobilized biotinylated extracellular domains of a human cell receptor of SARS-CoV-2, preferably the human angiotensin II converting enzyme type 2 (ACE2), affixed to the membrane using a biotin-streptavidin interaction. ACE2 is the human cell surface protein that serves as the receptor to which SARS-CoV-2 RBD binds to initiate infection.

If neutralizing antibodies are bound to the RBD, the labeled RBD fragments will not be able to bind to the immobilized ACE2 and will continue diffusing past the test line. If neutralizing antibodies are not present, the labeled RBD fragments will then be able to bind to the immobilized ACE2 on the test line, causing a build-up of gold nanoparticles and horseradish peroxidase.

Any RBD fragments not bound by neutralizing antibodies will continue diffusing across the membrane and encounter the control line where antibodies recognizing horseradish peroxidase will be immobilized. These antibodies will bind to the horseradish peroxidase that is labeling the RBD fragments, causing a build-up of both gold nanoparticles and horseradish peroxidase.

Results of the test can be visualized by the naked eye either looking for build-up of gold particles indicated by a red color, or by adding tetramethylbenzidine (TMB) and looking for precipitation of product as blue color.

If the patient has high-titer neutralizing antibodies, the test will demonstrate only 1 colored line, at the control line. If the patient has low-titer neutralizing antibodies, the test will demonstrate 2 colored lines, at both the control and test lines. If the patient has no neutralizing antibodies, the test will demonstrate only 1 colored line, at the test line.

The test provides test results with an accuracy of about 90% or greater, preferably about 95% or greater, preferably about 99% or greater and preferably about 100%.

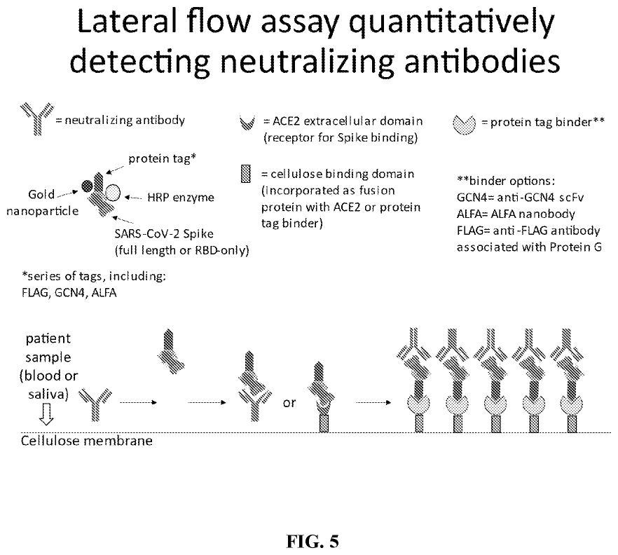

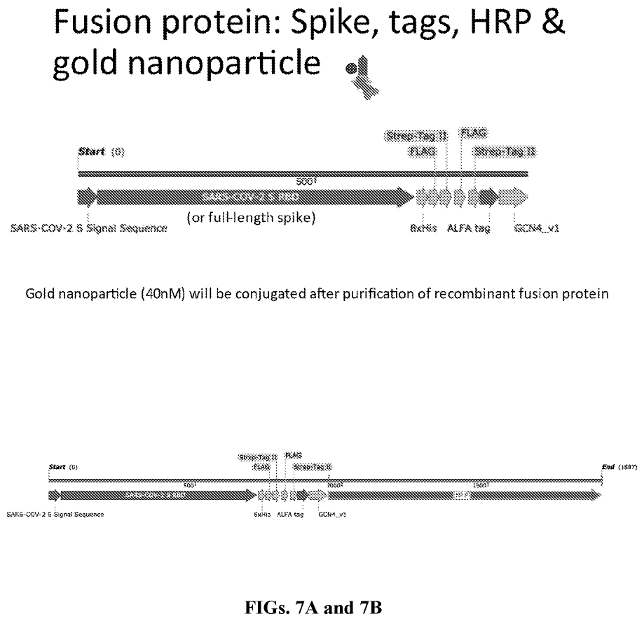

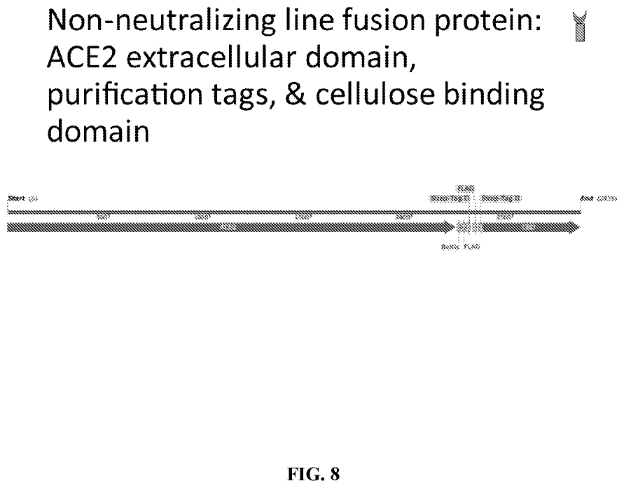

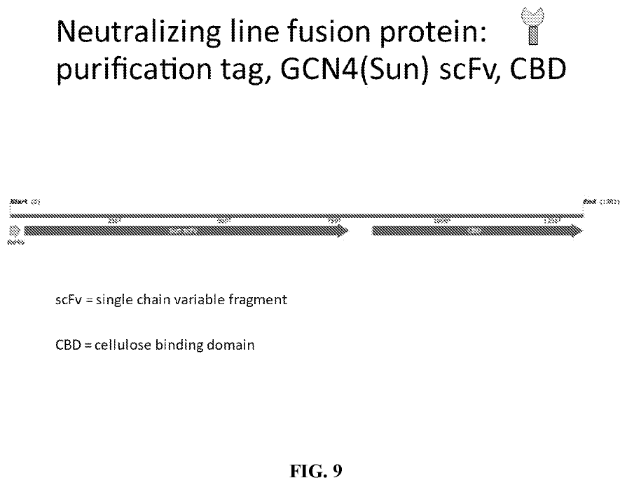

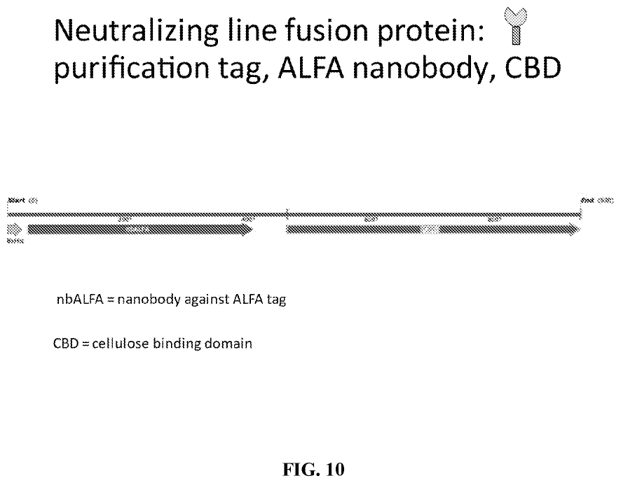

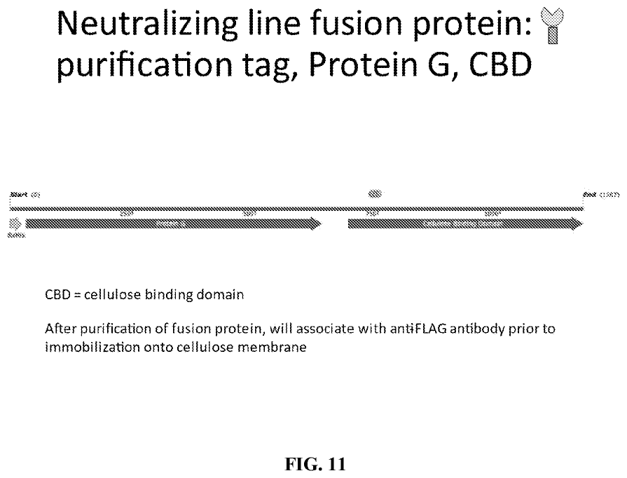

Another embodiment of the assay is shown in FIGS. 5 to 11. In these embodiments, the test requires a single drop of blood (or other biological sample) deposited onto one side of a test strip, preferably a cellulose or nitrocellulose membrane. The serum from the blood then diffuses across the membrane. Serum first interacts with a portion of the membrane impregnated with a fusion protein comprising a viral ligand of SARS-CoV-2, preferably solubilized receptor binding domain (RBD) fragments from the SARS-CoV-2 spike protein fused to one or more protein tags. The fusion proteins are preferably labeled with one or more detectable labels. Preferably the one or more detectable labels are gold nanoparticles and the horseradish peroxidase enzyme.

It is understood that a fusion protein comprising the full-length SARS-CoV-2 spike protein comprising receptor binding domains that target human cells for infection may also be used instead of, or in addition to, the solubilized RBD fragments of SARS-CovV-2. The full-length SARS-CoV-2 fusion protein may also be labeled with a detectable label in the same manner the RBD fragments of SARS-CoV-2 are labeled.

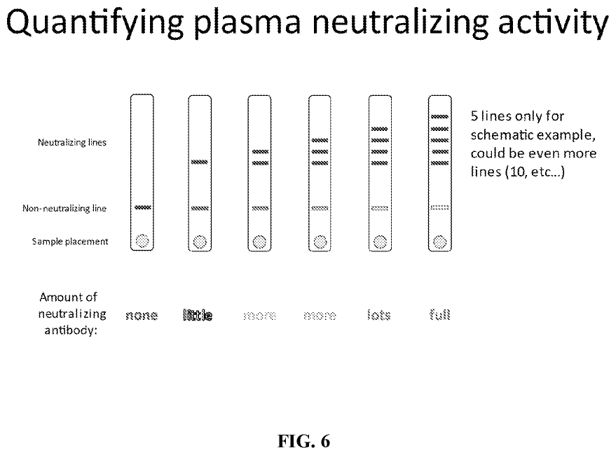

As shown in FIGS. 5 and 6, any neutralizing antibodies in the patient's serum will bind to the soluble RBD fragments, as true neutralizing antibodies will bind to the SARS-CoV-2 RBD and disrupt the virus's ability to attach to and infect human cells. The diffusion of serum across the membrane will then carry both unbound and antibody-bound labeled RBD fusion protein across the membrane by lateral flow, and then reach the test line.

In one embodiment, the test line contains immobilized extracellular domains of a human cell receptor of SARS-CoV-2, preferably the human angiotensin II converting enzyme type 2 (ACE2), affixed to the membrane optionally using a cellulose binding domain fused to the ACE2 if the test strip comprises cellulose. ACE2 is the human cell surface protein that serves as the receptor to which SARS-CoV-2 RBD binds to initiate infection. FIGS. 5-10 provide the cloning construct maps for the various fusion proteins used in this embodiment.

If neutralizing antibodies are bound to the RBD, the labeled RBD fusion protein will not be able to bind to the immobilized ACE2 and will continue diffusing past the test line. If neutralizing antibodies are not present, the labeled RBD fragments will then be able to bind to the immobilized ACE2 on the test line, causing a build-up of gold nanoparticles and horseradish peroxidase.

Any RBD fusion protein not bound by neutralizing antibodies will continue diffusing across the membrane and encounter the control line or neutralizing line where a protein tag binder will be immobilized. The protein tag binder will bind the protein tag on the RBD fusion protein, causing a build-up of both gold nanoparticles and horseradish peroxidase.

Results of the test can be visualized by naked eye either looking for build-up of gold particles indicated by a red color, or by adding tetramethylbenzidine (TMB) and looking for precipitation of product as blue color.

If the patient has high-titer neutralizing antibodies, the test will show color at the one or more control lines of the test strip. If the patient has low-titer neutralizing antibodies, the test will demonstrate color at both the test line and the one or more control lines. If the patient has no neutralizing antibodies, the test will demonstrate only one colored line, at the test line.

The test provides test results with an accuracy of about 90% or greater, preferably about 95% or greater, preferably about 99% or greater and preferably about 100%.

The present invention is not limited to assays identifying neutralizing antibodies against SARS-CoV-2 only. Using the same principals of the above-described invention, the invention also provides a lateral flow test strip for detecting the presence of neutralizing antibodies against a target virus in a blood or serum sample from a patient, wherein the test strip comprises: a sample application region; a conjugation region wherein the conjugation region comprises a plurality of viral ligands or fragments thereof derived from the target virus wherein the viral ligands or fragments thereof are capable of binding a cell receptor on a host cell in a patient and infecting the host cell with target viral material, wherein the viral ligand is optionally a fusion protein labelled with a protein tag and are further fused to and/or conjugated with a detection label that is visible to the naked eye, and preferably at least one detectable label is an enzyme detection label; a test line comprising a plurality of cell receptors immobilized at the test line wherein the receptors are derived from the host cell and are capable of being bound by the solubilized receptor or fragments thereof of the target virus; and a control line comprising molecules capable of binding a detectable label or protein tag. Preferably the patient is a human patient and the cell receptors are human cell receptors.

Examples of other viral infections, viral ligands or fragments thereof and human host cell receptors are described in Table 1.

TABLE 1

| Infection | Viral ligand | Human receptor |
|---|---|---|
| SARS | SARS-CoV-1 spike protein | ACE2 |
| MERS | MERS-CoV spike protein | DPP4 |
| Ebola | Ebola virus membrane phosphatidyl serine | TIM-1 |
| Marburg | Marburg virus membrane phosphatidyl serine | TIM-1 |
| Lassa fever | Lassa fever virus glycoprotein (spike) | alpha-dystroglycan |
| Dengue | Dengue virus envelope E protein | heparan sulfate |
| | | glycosaminoglycan |
| | | (GAG) |
| Japanese | Japanese encephalitis virus envelope E protein | HSP70 |
| Encephalitis | | |
| Yellow fever | Yellow fever virus envelope E protein | unknown . . . |
| Zika | Zika virus envelope E protein | TIM-1 |
| West Nile | West Nile virus envelop E protein | DC-SIGN |
| Triple E | Eastern equine encephalitis virus E2 | Heparan sulfate |
| | protein | |
| Malaria | Plasmodium sporozoite TRAP | Integrin alphavbeta3 |
| | Plasmodium falciparum PfRH5 | Basigin |
| | Plasmodium vivax Duffy binding protein | Duffy antigen |

It is understood that binding fragments of the viral ligands of the full length proteins listed in Table 1 are suitable for use in the invention It is also understood that other human cell receptors may be used by the viruses and any such cell receptors that bind a viral ligand may be used in the assays according to the invention.

The invention is illustrated by the following non-limiting examples.

### EXEMPLIFICATION

### Example 1: Design of Plasma Protein Internal Control for SARS-CoV-2 Neutralizing Antibody Quantitative Lateral Flow Assay

A sample (either pin-prick of blood or abrasive gum swab) was placed into incubation with soluble gold-conjugated proteins (RBD) in binding/running buffer for a brief incubation period. The incubation period allows the neutralizing antibodies (nAbs), if present, to bind to the gold-conjugated RBD and also allows a control plasma protein to bind to gold-conjugated monoclonal antibody. The control plasma protein is a protein with highly stable concentration and albumin was tested. After the brief incubation, the mixture is applied to the test strip. RBD, if not neutralized, will bind to immobilized ACE2 (recombinant extracellular domain of human ACE2) at the test line. Neutralized RBD will continue flowing. The plasma protein control is always caught at the control line which includes an immobilized polyclonal antibody against the plasma protein. Thus, the ratio of signal at the control over the signal at the test line equated essentially to nAb titer in the biological sample. These signals (at control line and test line) can easily be measured with a smartphone camera and app.

In the field, it may not be possible to obtain a precise volume of either a pin-prick of blood or abrasive gum swab. If an inexact volume of sample is input into the test, then the absolute amount of the nAbs in the test will be variable even if taken as replicates from the same patient on the same day. Thus, having a highly constant plasma protein internal control will be of use in normalizing the amount of nAb input into the assay and normalizing plasma concentration. Thus, no matter how variable the sample volume input is, the results can be normalized to a plasma titer for nAb. Visualization of plasma control protein at the control line via antibody-sandwich methodology serves as control.

We have determined that:
1) It is possible to have gold-conjugated RBD bind to an immobilized ACE2 extracellular domain and give a strong enough visual signal at the test line in the absence of neutralizing antibody; and
2) There is a visually discernible decrease in signal in immobilized ACE2 test line if neutralizing antibody is present.
The following running buffers have been tested, all comprising 0.1% Brondiox and further comprising:
   3H-11=3% Triton X305/5% C-BL/1% NaCl/1×TE
   3H-10=5% X305/5% C-BL/0.9% NaCl/1×TE
   3H-9=5% X305/2% sucrose (suc)/5% C-BL/0.5% NaCl/1×TE
   3H-8=5% X305/2% suc/5% C-CL/0.9% NaCl/1×TE
   3H-7=4% X305/2% suc/5% C-BL/0.9% NaCl/1×TE
   3H-6=3% X305/2% suc/5% C-BL/0.9% NaCl/1×TE
   3H-5=3% X305/5% suc/5% C-BL/1% NaCl/1×TE
   3H-4=4% X305/5% suc/1% C-BL/1% NaCl/1×TE
   3H-3=3% X305/10% suc/1% C-BL/1% NaCl/1×TE
   3H-2=3% X305/5% suc/1% C-BL/1×PBS
   3H-1=3% X305/5% suc/1% C-BL/1% NaCl/1×TE 3H=1× Diluent-3H [10×=25% X305/15% suc/5% C-BL/10×TE]+0.9% NaCl

SEQUENCE LISTING

Seq ID NO: 1-SARScoV2RBD-8xHis-FLAG-Strep-FLAG-
Strep-ALFA-GCN4-HRP
MFVFLVLLPLVSSQRVQPTESIVRFPNITNLCPFGEVFNATRFASVYAW
NRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNY
LYRLFRKSNLKPFERDISTEIYQAGSTPCNGVEGFNCYFPLQSYGFQPT
NGVGYQPYRVVVLSFELLHAPATVCGPKKSTNLVKNKCVNFSRHHHHHH
HHDYKDDDDKWSHPQFEKGGDYKDDDDKSAWSHPQFEKSRLEEELRRRL
TELLPKNYHLENEVARLKKLVGERMQLTPTFYDNSCPNVSNIVRDTIVN
ELRSDPRIAASILRLHFHDCFVNGCDASILLDNTTSFRTEKDAFGNANS
ARGFPVIDRMKAAVESACPRTVSCADLLTIAAQQSVTLAGGPSWRVPLG
RRDSLQAFLDLANANLPAPFFTLPQLKDSFRNVGLNRSSDLVALSGGHT
FGKSQCRFIMDRLYNFSNTGLPDPTLNTTYLQTLRGLCPLNGNLSALVD
FDLRTPTIFDNKYYVNLEEQKGLIQSDQELFSSPNATDTIPLVRSFANS
TQTFFNAFVEAMDRMGNITPLTGTQGQIRLNCRVVNSNSDL*
(SEQ ID NO: 1)
SEQ ID NO: 2-SARScoV2FullLengthSpike-8xHis-FLAG-
Strep-FLAG-Strep-ALFA-GCN4-HRP:
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPASVAS
QSIIAYTMSLGAENSVAYSNNSIAIPTNFTISVTTEILPVSMTKTSVDC
TMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQ
YGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKI
QDSLSSTASALGKLQDVVNQNAQALNTLVKQLSSNFGAISSVLNDILSR
LDPPEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNI
QKEIDRLNEVAKNLNESLIDLQELGKYEQYIKWPSRHHHHHHHHDYKDD
DDKWSHPQFEKGGDYKDDDDKSAWSHPQFEKSRLEEELRRRLTELLPKN
YHLENEVARLKKLVGERMQLTPTFYDNSCPNVSNIVRDTIVNELRSDPR
IAASILRLHFHDCFVNGCDASILLDNTTSFRTEKDAFGNANSARGFPVI
DRMKAAVESACPRTVSCADLLTIAAQQSVTLAGGPSWRVPLGRRDSLQA
FLDLANANLPAPFFTLPQLKDSFRNVGLNRSSDLVALSGGHTFGKSQCR
FIMDRLYNFSNTGLPDPTLNTTYLQTLRGLCPLNGNLSALVDFDLRTPT
IFDNKYYVNLEEQKGLIQSDQELFSSPNATDTIPLVRSFANSTQTFFNA
FVEAMDRMGNITPLTGTQGQIRLNCRVVNSNSDL*
(SEQ ID NO: 2)
SEQ ID NO: 3 ACE2-8xHis-FLAG-Strep-FLAG-Strep-CBD
MSSSSWLLLSLVAVTAAQSTIEEQAKTFLDKFNHEAEDLFYQSSLASWN
YNTNITEENVQNMNNAGDKWSAFLKEQSTLAQMYPLQEIQNLTVKLQLQ
ALQQNGSSVLSEDKSKRLNTILNTMSTIYSTGKVCNPDNPQECLLLEPG
LNEIMANSLDYNERLWAWESWRSEVGKQLRPLYEEYVVLKNEMARANHY
EDYGDYWRGDYEVNGVDGYDYSRGQLIEDVEHTFEEIKPLYEHLHAYVR
AKLMNAYPSYISPIGCLPAHLLGDMWGRFWTNLYSLTVPFGQKPNIDVT
DAMVDQAWDAQRIFKEAEKFFVSVGLPNMTQGFWENSMLTDPGNVQKAV
CHPTAWDLGKGDFRILMCTKVTMDDFLTAHHEMGHIQYDMAYAAQPFLL
RNGANEGFHEAVGEIMSLSAATPKHLKSIGLLSPDFQEDNETEINFLLK
QALTIVGTLPFTYMLEKWRWMVFKGEIPKDQWMKKWWEMKREIVGVVEP
VPHDETYCDPASLFHVSNDYSFIRYYTRTLYQFQFQEALCQAAKHEGPL
HKCDISNSTEAGQKLFNMLRLGKSEPWTLALENVVGAKNMNVRPLLNYF
EPLFTWLKDQNKNSFVGWSTDWSPYADQSIKVRISLKSALGDKAYEWND
NEMYLFRSSVAYAMRQYFLKVKNQMILFGEEDVRVANLKPRISFNFFVT
APKNVSDIIPRTEVEKAIRMSRSRINDAFRLNDNSLEFLGIQPTLGPPN
QPPVSSRHHHHHHHHDYKDDDDKWSHPQFEKGGDYKDDDDKSAWSHPQF
EKPVSGNLKVEFYNSNPSDTTNSINPQFKVTNTGSSAIDLSKLTLRYYY
TVDGQKDQTFWCDHAAIIGSNGSYNGITSNVKGTFVKMSSSTNNADTYL
EISFTGGTLEPGAHVHIQGRFAKNDWSNYTQSNDYSFKSASQFVEWDQV
TPYLNGVLVWGKEP* (SEQ ID NO: 3)
SEQ ID NO: 4 8xHis-GCN4scFv-CBD
MHHHHHHHHGGGMGPDAVVTQESALTTSPGETVTLTCRSSTGAVTTSNY
ASWVQEKPDHLFTGLIGGTNNRAPGVPARFSGSLIGDKAALTITGAQTE
DEAIYFCALWYSNHWVFGGGTKLTVLGGGGGSGGGGSGGGGSSGGGSEV
KLVESGPGLVAPSQSLSITCTVSGFSLTDYGVNWVRQSPGKGLEWLGVI
WGDGITDYNSALKSRLSVTKDNSKSQVFLKMNSLQSGDSARYYCVTGLF
DYWGQGTTLTVSSGSGGGSGGGSGGGSGGSGPVSGNLKVEFYNSNPSDT
TNSINPQFKVTNTGSSAIDLSKLTLRYYYTVDGQKDQTFWCDHAAIIGS
NGSYNGITSNVKGTFVKMSSSTNNADTYLEISFTGGTLEPGAHVHIQGR
FAKNDWSNYTQSNDYSFKSASQFVEWDQVTPYLNGVLVWGKEP*
(SEQ ID NO: 4)
SEQ ID NO: 5 8xHis-ALFAnanobody-CBD
MHHHHHHHHGGGEVQLQESGGGLVQPGGSLRLSCTASGVTISALNAMAM
GWYRQAPGERRVMVAAVSERGNAMYRESVQGRFTVTRDFTNKMVSLQMD
NLKPEDTAVYYCHVLEDRVDSFHDYWGQGTQVTVSSGSGGGSGGGSGGG
SGGSGPVSGNLKVEFYNSNPSDTTNSINPQFKVTNTGSSAIDLSKLTLR
YYYTVDGQKDQTFWCDHAAIIGSNGSYNGITSNVKGTFVKMSSSTNNAD
TYLEISFTGGTLEPGAHVHIQGRFAKNDWSNYTQSNDYSFKSASQFVEW
DQVTPYLNGVLVWGKEP (SEQ ID NO: 5)
SEQ ID NO: 6 8xHis-ProteinG-CBD
MHHHHHHHHGGGMLPKTDTYKLILNGKTLKGETTTEAVDAATAEKVFKQ
YANDNGVDGEWTYDDATKTFTVTEKPEVIDASELTPAVTTYKLVINGKT
LKGETTTEAVDAATAEKVFKQYANDNGVDGEWTYDDATKTFTVTEKPEV
IDASELTPAVTTYKLVINGKTLKGETTTKAVDAETAEKAFKQYANDNGV
DGVWTYDDATKTFTVTEGSGGGSGGGSGGGSGGSGPVSGNLKVEFYNSN
PSDTTNSINPQFKVTNTGSSAIDLSKLTLRYYYTVDGQKDQTFWCDHAA
IIGSNGSYNGITSNVKGTFVKMSSSTNNADTYLEISFTGGTLEPGAHVH
IQGRF AKNDWSNYTQSNDYSFKSASQFVEWDQVTPYLNGVLVWGKEP
(SEQ ID NO: 6).

### REFERENCES

1. Chu, D. K. W. et al. Molecular Diagnosis of a Novel Coronavirus (2019-nCoV) Causing an Outbreak of Pneumonia. Clin Chem, doi:10.1093/clinchem/hvaa029 (2020).
2. Corman, V. M. et al. Detection of 2019 novel coronavirus (2019-nCoV) by real-time RT-PCR. Euro Surveill 25, doi:10.2807/1560-7917.ES.2020.25.3.2000045 (2020).
3. Wang, W. et al. Detection of SARS-CoV-2 in Different Types of Clinical Specimens. JAMA doi:10.1001/jama.2020.3786 (2020).
4. Xu, Y. et al. Characteristics of pediatric SARS-CoV-2 infection and potential evidence for persistent fecal viral shedding. Nature Medicine (2020).
5. Xie, C. et al. Comparison of different samples for 2019 novel coronavirus detection by nucleic acid amplification tests. Int J Infect Dis 93, 264-267, doi:10.1016/j.ijid.2020.02.050 (2020).
6. Whoriskey, P. & Satija, N. How U.S. coronavirus testing stalled: flawed tests, red tape and resistance to using the millions of tests produced by the WHO. Washington Post (2020).
7. Coronavirus (COVID-19) Update: FDA Issues first Emergency Use Authorization for Point of Care Diagnostic. FDA News Release (2020).
8. Chan-Yeung, M. Severe acute respiratory syndrome (SARS) and healthcare workers. Int J Occup Environ Health 10, 421-427, doi:10.1179/oeh.2004.10.4.421 (2004).
9. Chen, Y. C. et al. Infection control and SARS transmission among healthcare workers, Taiwan. Emerg Infect Dis 10, 895-898, doi:10.3201/eid1005.030777 (2004).
10. Amanat, F. et al. A serologic assay to detect SARS-CoV-2 seroconversion in humans. MedRxiv (2020).
11. Li, Z. et al. Development and Clinical Application of A Rapid IgM-IgG Combined Antibody Test for SARS-CoV-2 Infection Diagnosis. J Med Virol, doi:10.1002/jmv.25727 (2020).

All references, articles, patent applications, patent publications and patents are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1-130. (canceled)

131. A lateral flow test strip for
(I) detecting the presence or titer of neutralizing antibodies (nAbs) to SARS-CoV-2 in a biological sample from a patient, wherein the test strip comprises: a sample application region; a test line comprising an extracellular domain of the human angiotensin II converting enzyme type 2 (ACE2) receptor immobilized at the test line; and optionally a control line capable of specifically binding to a control protein in the sample; wherein the test line and the optional control line are located downstream from the sample application region; wherein the biological sample is contacted with a SARS-CoV-2 antigen before or after application to the sample application region thus forming a treated sample, wherein the SARS-CoV-2 antigen is labeled with a first detectable label, and wherein the strip is configured such that the presence or titer of nAbs is inversely proportional to the amount of the first detectable label captured at the test line; or
(II) detecting the presence of neutralizing antibodies to SARS-CoV-2 in a blood or serum sample from a patient, wherein the test strip comprises: a sample application region; a conjugation region wherein the conjugation region comprises at least one fusion protein comprising a protein or protein fragment derived from SARS-CoV-2 fused to a protein tag wherein the fusion protein further comprises a detection label that is visible to the naked eye; a test line comprising at least one extracellular domain of the human angiotensin II converting enzyme type 2 (ACE2) receptor immobilized at the test line; and one or more control lines comprising at least one protein tag binder capable of specifically binding the protein tag of the fusion protein; or
(III) detecting the presence of neutralizing antibodies against a target virus in a blood or serum sample from a patient, wherein the test strip comprises: a sample application region; a conjugation region comprising at least one fusion protein wherein the fusion protein comprises a protein or protein fragment derived from all or a portion of a viral ligand of the target virus wherein the viral ligand is capable of binding a cell receptor on a host cell in a patient and infecting the host cell, fused to a protein tag, a detection label that is visible to the naked eye or both; a test line at least one cell receptor or binding fragment thereof immobilized at the test line, wherein the receptor is derived from the host cell and are capable of being bound by the solubilized receptor or fragments thereof of the target virus; and a control line comprising at least one immobilized anti-conjugate molecule capable of specifically binding to some portion of the fusion protein; or
(IV) detecting the presence of neutralizing antibodies to SARS-CoV-2 in a blood or serum sample from a patient, wherein the test strip comprises: a sample application region; a conjugation region wherein the conjugation region comprises a plurality of solubilized receptor binding domain (RBD) fragments from the SARS-CoV-2 spike protein wherein the solubilized RBD fragments are labeled with one or more detection labels that are visible to the naked eye wherein at least one detection label is an enzyme detection label; a test line comprising a plurality of extracellular domains of the human angiotensin II converting enzyme type 2 (ACE2) immobilized at test line by the interaction of biotin with streptavidin affixed at the test line; and a control line comprising immobilized antibodies capable of binding the enzyme detection label on the solubilized RBD fragments; or
(V) detecting the presence of neutralizing antibodies against a target virus in a blood or serum sample from a patient, wherein the test strip comprises: a sample application region; a conjugation region wherein the conjugation region comprises a plurality of viral ligands or fragments thereof derived from the target virus wherein the viral ligands or fragments thereof are capable of binding a cell receptor on a host cell in a patient and infecting the host cell with target viral material, wherein the viral ligand or fragments thereof are labeled with one or more detection labels that are visible to the naked eye, and wherein at least one detectable label is an enzyme detection label; a test line comprising a plurality of biotinylated cell receptor immobilized at the test line by the interaction of biotin with streptavidin affixed at the test line wherein the receptors are derived from the host cell and are capable of being bound by the solubilized receptor or fragments thereof of the target virus; and a control line comprising antibodies capable of binding the enzyme detection label.

132. The lateral flow test strip of claim 131, wherein the strip further comprises a conjugation region, wherein the conjugation region comprises the SARS-CoV-2 antigen and wherein the conjugation region is downstream of the sample application region and upstream of the test line.

133. The lateral flow test strip of claim 132 wherein the control line comprises an immobilized first antibody with antigenic specificity for the control protein, wherein the conjugation region further comprises a second antibody with antigenic specific for the control protein, and wherein the second antibody is labeled with a second detectable label.

134. The lateral flow test strip of claim 131, wherein (i) the detection label comprises horseradish peroxidase (HRP); the detection label comprises gold nanoparticles and HRP; (iii) the detection label comprises nanoparticles selected from the group consisting of carbon-based nanoparticles, quantum dots, lanthanides and up-converting phosphor; (iv) the test strip comprises one or more control lines comprising at least one protein tag binder capable of specifically binding the protein tag of the fusion protein; (v) the fusion protein comprising a protein or protein fragment derived from SARS-CoV-2 comprises the amino acid sequence of SEQ ID NO: 1 or SEQ ID NO: 2; (vi) the fusion protein comprising the ACE2 extracellular domain fused to a cellulose binding domain comprises the amino acid sequence of SEQ ID NO: 3; and/or (vii) the fusion protein comprising the protein tag binder fused to a cellulose binding domain comprises the amino acid sequence of SEQ ID NO: 4, SEQ ID NO: 5 or SEQ ID NO: 6.

135. A diagnostic kit comprising the lateral flow test strip of claim 131, wherein the kit further comprises the SARS-CoV-2 antigen labeled with a first detectable label.

136. The diagnostic kit of claim 135, wherein the kit further comprises one or more of (a) an antibody with antigenic specificity for a control protein, wherein the antibody is labeled with a second detectable label; (b) a buffer for incubating the biological sample with the SARS-CoV-2 antigen; (c) a sample collection device for the biological sample; (d) a substrate of HRP, optionally wherein the substrate is TMB (3,3′,5,5′-tetramethylbenzidine), OPD (o-phenylenediamine dihydrochloride) or BTS (2,2′-Azinobis [3-ethylbenzothiazoline-6-sulfonic acid]-diammonium salt); and/or (e) a fusion protein comprising the amino acid sequence of ny one of SEQ ID NO: 1-6.

137. A method of
(I) detecting the presence or titer of neutralizing antibodies (nAbs) to SARS-CoV-2 in a biological sample of a patient, comprising the steps of (a) contacting the biological sample with a SARS-CoV-2 antigen for a time sufficient for binding of nAbs present in the sample to bind the SARS-CoV-2 antigen thus forming a treated sample, wherein the SARS-CoV-2 antigen is labeled with a first detectable label; wherein the contacting step occurs before or after application to the sample application region; (b) applying the sample or the treated sample to the sample application region of the test strip of claim 131 so as to permit flow of the test sample from the sample application region to the test line and optionally thereafter to the control line; and (c) detecting the first detectable label at the test line and optionally detecting the second detectable label at the control line; wherein the amount of the first detectable label at the test line is inversely proportional to the titer of nAbs in the sample; or
(II) detecting the presence of neutralizing antibodies to SARS-CoV-2 in the blood or serum of a patient, comprising the steps of:
(a) applying a blood or serum sample from the patient to the sample application region of the lateral flow test strip of claim 131 so as to permit flow of the sample from sample application region to the conjugation region and then to the test line and thereafter to the control line;
(b) detecting the presence or absence of detectable label at the test line and the presence of absence of detectable label at the control line by viewing the presence or absence of the detectable label with the naked eye;
(c) wherein the presence of the detectable label at the test line and the absence of the detectable label at the control line is indicative of the absence of neutralizing antibodies against SARS-CoV-2 in the blood or serum of the patient, and wherein the presence of detectable label at the test line and the presence of detectable label at the control line indicate a low titer of neutralizing antibodies in the blood or serum of the patient, and wherein the absence of detectable label at the test line and the presence of detectable label at the control line is indicated of a high titer of neutralizing antibodies against SARS-CoV-2 in the blood or serum of the patient; or
(III) detecting the presence of neutralizing antibodies to SARS-CoV-2 in the blood or serum of a patient, comprising the steps of:
(a) applying a blood or serum sample from the patient to the sample application region of the lateral flow test strip of claim 131 so as to permit flow of the sample from sample application region to the conjugation region and then to the test line and thereafter to the control line;
(b) detecting the presence or absence of detectable label at the test line and the presence of absence of detectable label at the control line by viewing the presence or absence of the detectable label with the naked eye;
(c) wherein the presence of the detectable label at the test line and the absence of the detectable label at the control line is indicative of the absence of neutralizing antibodies against SARS-CoV-2 in the blood or serum of the patient, and wherein the presence of detectable label at the test line and the presence of detectable label at the control line indicate a low titer of neutralizing antibodies in the blood or serum of the patient, and wherein the absence of detectable label at the test line and the presence of detectable label at the control line is indicated of a high titer of neutralizing antibodies against SARS-CoV-2 in the blood or serum of the patient.

138. The method of claim 137, wherein the test strip further comprises a first antibody with antigenic specificity for the control protein immobilized at the control line.

139. The method of claim 137, wherein the biological sample is further contacted with a second antibody with antigenic specificity for the protein control before or after application to the sample application region, wherein the second antibody with antigenic specificity for the protein control is labeled with a second detectable label.

140. The method of claim 137, wherein (i) the protein control is albumin; (ii) the first antibody with antigenic specificity for the protein control is a polyclonal antibody; (iii) the second antibody with antigenic specific for the protein control is a monoclonal antibody; (iv) the first detectable label is a gold nanoparticle; and/or (v) the second detectable label is a gold nanoparticle.

141. The method of claim 137, comprising measuring the amount of label at the test line, and measuring the amount of label at the control line, wherein the ratio of the amount of first detectable label at the test line to the amount of second detectable label at the control line is inversely proportional to the titer of nAbs in the sample.

142. The method of claim 137, wherein (i) the sample is further contacted with a second antibody with antigenic specificity for the control protein before or after application to the sample application region; (ii) the SARS-CoV-2 antigen comprises all or a portion of the spike protein; (iii) the SARS-CoV-2 antigen comprises all or a portion of the receptor binding domain (RBD) of the spike protein; (iv) the SARS-CoV-2 antigen is recombinant; (v) the biological sample is a blood sample, a serum sample, a saliva sample, or an abrasive gum swab; and/or (vi) the strip further comprises a conjugation region, wherein the conjugation region comprises the SARS-CoV-2 antigen and wherein the conjugation region is downstream of the sample application region and upstream of the test line.

143. The method of claim 137, wherein (i) at least one detectable label comprises gold nanoparticles; (ii) the detectable enzyme label is HRP; (iii) the detectable label comprises HRP and gold nanoparticles; (iv) the detecting the presence or absence of detectable label at the test line and the control line comprises the step of viewing the formation of a red color with the naked eye; (v) the method further comprises applying a substrate of HRP to the test line and the control line wherein the substrate is TMB and viewing with the naked eye the formation of a blue color; (vi) detecting the presence or absence of detectable label at the test line and the control line comprises the step of viewing the formation of a red color with the naked eye; (vii) the method further comprises applying a substrate of HRP to the test line and the control line and viewing with the naked eye the formation of a blue color; and/or (viii) the substrate of HRP is TMB (3,3′,5,5′-tetramethylbenzidine).

144. A fusion protein comprising an amino acid selected from the group consisting of: SEQ ID NO: 1, SEQ ID NO: 2, SEQ ID NO: 3, SEQ ID NO: 4, SEQ ID NO: 5 and SEQ ID NO: 6.


SEQUENCE LISTING (6 sequences)

SEQ ID NO: 1 - protein, 629 aa, Artificial Sequence
  Synthetic
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFSRHHHHHHHHDYKDDDDKWSHPQFEKGGDYKDDDDKSAWSHPQFEKSRLEEELRRRLTELLPKNYHLENEVARLKKLVGERMQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKSQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSDL

SEQ ID NO: 2 - protein, 1602 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRHHHHHHHHDYKDDDDKWSHPQFEKGGDYKDDDDKSAWSHPQFEKSRLEEELRRRLTELLPKNYHLENEVARLKKLVGERMQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKSQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSDL

SEQ ID NO: 3 - protein, 945 aa, Artificial Sequence
  Synthetic
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSRHHHHHHHHDYKDDDDKWSHPQFEKGGDYKDDDDKSAWSHPQFEKPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVHIQGRFAKNDWSNYTQSNDYSFKSASQFVEWDQVTPYLNGVLVWGKEP

SEQ ID NO: 4 - protein, 435 aa, Artificial Sequence
  Synthetic
MHHHHHHHHGGGMGPDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSSGGGSEVKLVESGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSGSGGGSGGGSGGGSGGSGPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVHIQGRFAKNDWSNYTQSNDYSFKSASQFVEWDQVTPYLNGVLVWGKEP

SEQ ID NO: 5 - protein, 311 aa, Artificial Sequence
  Synthetic
MHHHHHHHHGGGEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGSGGGSGGGSGGGSGGSGPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVHIQGRFAKNDWSNYTQSNDYSFKSASQFVEWDQVTPYLNGVLVWGKEP

SEQ ID NO: 6 - protein, 390 aa, Artificial Sequence
  Synthetic
MHHHHHHHHGGGMLPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEGSGGGSGGGSGGGSGGSGPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVHIQGRFAKNDWSNYTQSNDYSFKSASQFVEWDQVTPYLNGVLVWGKEP